(19) United States
(12) Patent Application Publication
(10) Pub. No.: US 2025/0266146 A1
(43) Pub. Date: Aug. 21, 2025

(54) APPARATUS AND METHOD FOR CUSTOMIZED STRESS CARE

(71) Applicant: 40FY INC., Seoul (KR)

(72) Inventors: Seongjun MUN, Seoul (KR); Kyungjin LHO, Seoul (KR); Kiyoung SUNG, Seoul (KR); Woori MOON, Seongnam-si Gyeonggi-do (KR)

(21) Appl. No.: 19/194,938

(22) Filed: Apr. 30, 2025

Related U.S. Application Data
(63) Continuation-in-part of application No. PCT/KR2023/017280, filed on Nov. 1, 2023.

(30) Foreign Application Priority Data
Nov. 1, 2022 (KR) 10-2022-0143697
Nov. 1, 2022 (KR) 10-2022-0143718
Nov. 2, 2022 (KR) 10-2022-0144253

(51) Int. Cl. G16H 20/70; A61B 5/024; A61B 5/026; A61B 5/16; G16H 50/30; G16H 50/70

(52) U.S. Cl. CPC G16H 20/70; A61B 5/02405; A61B 5/0261; A61B 5/165; G16H 50/30; G16H 50/70

(57) ABSTRACT

In one aspect, the present invention relates to a stress measurement technology, and more specifically, to a facial blood flow-based stress measurement apparatus and method that provide a subject's resilience and high-risk group prediction results for stress by analyzing heart rate variability and stress levels based on facial images and psychological stress assessment results through a machine learning-based model.

### CROSS-REFERENCE TO RELATED APPLICATION(S)

This is a continuation-in-part application of International Patent Application No. PCT/KR2023/017280, filed Nov. 1, 2023 which claims priority from Korean Patent Application No. 10-2022-0143697, filed on Nov. 1, 2022, Korean Patent Application No. 10-2022-0144253, filed on Nov. 2, 2022 and Korean Patent Application No. 10-2022-0143718 filed on Nov. 1, 2022, which are hereby incorporated by reference in its entirety.

### FIELD

In one aspect, the present invention relates to stress measurement technology, and more specifically, to a facial blood flow-based stress measurement apparatus and method that provide a prediction result of resilience and a high-risk group for stress of a subject by analyzing heart rate variability and stress levels based on face video and psychological stress assessment results through a machine learning-based model.

In another aspect, the present invention relates to stress measurement technology, and more specifically, to a finger blood flow-based stress measurement apparatus and method that provide a prediction result of resilience and a high-risk group for stress of a subject by analyzing heart rate variability and stress levels measured from finger video and psychological stress assessment results collected from a survey through a machine learning-based model.

In yet another aspect, the present invention relates to stress measurement and care technology, and more specifically, to a user-customized stress care apparatus and method that can provide feedback for stress relief by analyzing human body video and stress questionnaire test result data to obtain stress measurement results and repeatedly measuring stress while proceeding with each care process of a stress management program accordingly.

### BACKGROUND

Heart Rate Variability (HRV) is a value that measures the temporal variation between heartbeats (heart rate) regulated by the sympathetic and parasympathetic nervous systems. HRV is a quantitative indicator that is critically considered when identifying the emotional response state of the body and is referenced for clinical interpretation of the autonomic nervous system's condition. Generally, when measuring HRV, a physical detection mechanism (electrocardiogram) that contacts the skin is used to collect accurate signals. However, electrocardiogram equipment is expensive, incurring costs, and it is challenging to measure conveniently in daily life.

Recently, technology has been developed to measure HRV by capturing face or finger videos and extracting the location of arterial blood flow close to the facial skin or the location of finger blood flow. Using the HRV and related indicators measured in this way, it is possible to identify the stress state corresponding to the body's emotional response state. However, there is a problem in that estimating the stress state using only HRV-related data is not accurate. Furthermore, while the method of measuring HRV using face or finger videos has the advantage of being almost cost-free and convenient for daily measurement, it may be somewhat less accurate compared to the method using an electrocardiogram. This makes it difficult to meet the needs of individuals who wish to accurately monitor their stress levels in everyday life.

In addition, although offline or online care programs for stress states are currently being provided, they fail to quantify and compare the actual therapeutic effects to offer optimal stress care programs tailored to individual users.

### SUMMARY

The present invention has been devised to solve the above-mentioned problems in one aspect, and an object of the present invention is to provide a stress measurement result with high predictability through non-contact measurement. Another object of the present invention is to enhance accessibility to repeated tests using a smartphone, thereby enabling the tracking of stress symptoms.

In another aspect, another object of the present invention is to provide an optimal stress care program by repeatedly measuring the stress state during stress care.

In yet another aspect, another object of the present invention is to present the amount of change in the stress measurement result as a numerical value during stress care, thereby allowing the therapeutic effect to be visually confirmed.

According to an embodiment of the present disclosure to solve the above-described problem, a method for measuring and managing stress performed by an apparatus for measuring and managing stress comprising at least one processor is disclosed. The method comprises: outputting survey items on a screen according to an interaction in which a camera recording button is input; generating stress and internal vulnerability test result data based on response contents to the survey items; extracting a blood flow location from a face video captured by a camera, and measuring a first heart rate interval based on first pixel data of the extracted location; analyzing a measurement result of measuring the first heart rate interval and calculating a first heart rate variability; determining a first stress level based on the calculated first heart rate variability; extracting a blood flow location from a finger video captured by the camera, and measuring a second heart rate interval based on second pixel data of the extracted location; analyzing a measurement result of measuring the second heart rate interval and calculating a second heart rate variability; determining a second stress level based on the calculated second heart rate variability; outputting a stress measurement result, which is a prediction result of resilience and a high-risk group for stress, based on the first heart rate variability, the second heart rate variability, the first stress level, the second stress level, and the stress and internal vulnerability test result data; and providing a stress management program comprising a plurality of care processes according to the stress measurement result.

In addition, the step of outputting a stress measurement result, which is a prediction result of resilience and a high-risk group for stress, based on the first heart rate variability, the second heart rate variability, the first stress level, the second stress level, and the stress and internal vulnerability test result data comprises: determining the stress measurement result by analyzing the first heart rate variability, the second heart rate variability, the first stress level, the second stress level, and the stress and internal vulnerability test result data through a machine learning-based model.

In addition, the step of outputting survey items on a screen according to an interaction in which a camera recording button is input comprises: starting capturing a user using the camera according to the interaction; outputting the survey items on the screen while the user is being captured; and terminating capturing of the user when a preset time related to a recording time has elapsed.

In addition, the step of terminating capturing of the user when a preset time related to a recording time has elapsed comprises: terminating capturing of the user when it is determined that 1 minute has elapsed since the interaction occurred.

In addition, each of the first heart rate variability and the second heart rate variability comprises a time domain index analyzed in a time domain and a frequency domain index analyzed in a frequency domain for heart rate interval data collected for a predetermined time, the time domain index comprises a SDNN (Standard Deviation of Normal-to-Normal intervals) and a RMSSD (Root Mean Square of Successive Differences between normal heartbeats), and the frequency domain index represents ratios occupied by VLF (Very low frequency: 0.0033 to 0.04 Hz band), LF (Low frequency: 0.04 to 0.15 Hz band), and HF (High frequency: 0.15 to 0.4 Hz band) in a frequency domain by frequency-converting a heart rate interval data.

In addition, the SDNN is determined based on a mathematical formula

$$SDNN = \sqrt{\frac{1}{N - 1} {{\sum }_{i = 1}^{N} [ Mean ( NN ) - {NN}_{i} ]}^{2}} ,$$

and the i and the N are natural numbers, and the NN is the first heart rate interval or the second heart rate interval, and the RMSSD is determined based on a mathematical formula

$$RMSSD = \sqrt{\frac{1}{N - 2} {{\sum }_{i = 2}^{N} [ Mean ( NN ) - {NN}_{i - 1} ]}^{2}} .$$

In addition, the step of providing a stress management program comprising a plurality of care processes according to the stress measurement result comprises: determining a care plan in which the plurality of care processes are provided—the care plan comprises first plan information for determining at least some care processes among the plurality of care processes and second plan information for determining an order in which the at least some care processes are provided—; providing the at least some care processes according to a result of the care plan; outputting a sub-stress measurement result according to each of the at least some care processes when each of the at least some care processes is terminated; calculating a change value between a first sub-stress measurement result measured first and a second sub-stress measurement result measured last when all of the at least some care processes are provided; and outputting the calculated change value.

In addition, the step of providing the at least some care processes according to a result of the care plan comprises: starting capturing of a user using the camera in conjunction with an operation of providing the at least some care processes, and wherein the step of calculating a change value between a first sub-stress measurement result measured first and a second sub-stress measurement result measured last when all of the at least some care processes are provided comprises: terminating capturing of the user when all of the at least some care processes are provided.

In addition, the step of determining a care plan in which the plurality of care processes are provided comprises: determining whether a past change value determined within a preset previous time period from a current point in time exists; and determining the care plan based on the past change value when it is determined that the past change value exists.

In addition, the step of determining the care plan based on the past change value when it is determined that the past change value exists comprises: determining the care plan based on the past change value when the past change value is equal to or greater than a predetermined threshold; and determining the care plan without using the past change value when the past change value is less than the predetermined threshold.

In addition, the step of determining the care plan based on the past change value when the past change value is equal to or greater than a predetermined threshold comprises: determining a past care provision history from which the past change value was calculated; and determining the care plan based on the past care provision history.

Also, an apparatus for measuring and managing stress is disclosed. The apparatus comprises: a stress survey unit configured to output survey items on a screen according to an interaction in which a camera recording button is input, and generate stress and internal vulnerability test result data based on response contents to the survey items; an image analysis unit configured to extract a blood flow location from a face video captured by a camera, measure a first heart rate interval based on first pixel data of the extracted location, extract a blood flow location from a finger video captured by the camera, and measure a second heart rate interval based on second pixel data of the extracted location; a computation unit configured to analyze a measurement result of measuring the first heart rate interval and calculate a first heart rate variability, and analyze a measurement result of measuring the second heart rate interval and calculate a second heart rate variability; a stress calculation unit configured to determine a first stress level based on the calculated first heart rate variability, and determine a second stress level based on the calculated second heart rate variability; an estimation unit configured to output a stress measurement result, which is a prediction result of resilience and a high-risk group for stress, based on the first heart rate variability, the second heart rate variability, the first stress level, the second stress level, and the stress and internal vulnerability test result data; and a care program providing unit configured to provide a stress management program comprising a plurality of care processes according to the stress measurement result.

### BRIEF DESCRIPTION OF THE FIGURES

FIG. 1 is an internal configuration diagram of a facial blood flow-based stress measurement apparatus according to the first embodiment of the present invention.

FIG. 2 is an internal configuration diagram of an estimation unit according to the first embodiment of the present invention.

FIG. 3 is a flowchart of a face recognition-based stress measurement method according to the first embodiment of the present invention.

FIG. 4 is a diagram showing the operation of a facial blood flow-based stress measurement apparatus connected to a network according to the second embodiment of the present invention.

FIG. 5 is a flowchart of a face recognition-based stress measurement method according to the second embodiment of the present invention.

FIG. 6 is an internal configuration diagram of a finger blood flow-based stress measurement apparatus according to the third embodiment of the present invention.

FIG. 7 is an internal configuration diagram of an estimation unit according to the third embodiment of the present invention.

FIG. 8 is a flowchart of a finger blood flow-based stress measurement method according to the third embodiment of the present invention.

FIG. 9 is a diagram showing the operation of a finger blood flow-based stress measurement apparatus connected to a network according to the fourth embodiment of the present invention.

FIG. 10 is a flowchart of a finger blood flow-based stress measurement method according to the fourth embodiment of the present invention.

FIG. 11 is a diagram showing a smartphone screen for finger blood flow-based stress measurement according to the present invention.

FIG. 12 is an internal configuration diagram of a user-customized stress care apparatus according to the fifth embodiment of the present invention.

FIG. 13 is an internal configuration diagram of a stress measurement unit according to the fifth embodiment of the present invention.

FIG. 14 is an internal configuration diagram of an estimation unit according to the fifth embodiment of the present invention.

FIG. 15 is a flowchart of a user-customized stress care method according to the fifth embodiment of the present invention.

FIG. 16 is a flowchart showing the process of measuring stress in a user-customized stress care method according to the fifth embodiment of the present invention.

FIG. 17 is a diagram showing the operation of a user-customized stress care apparatus connected to a network according to the sixth embodiment of the present invention.

FIG. 18 is a flowchart showing the process of measuring stress in a user-customized stress care method according to the sixth embodiment of the present invention.

FIG. 19 shows another sequence process of a stress measurement method according to some embodiments of the present invention.

### DETAILED DESCRIPTION

Below, the embodiments of the present invention will be described in detail with reference to the accompanying drawings so that those skilled in the art to which the present invention pertains can easily implement the invention. However, the present invention can be implemented in various different forms and is not limited to the embodiments described herein. Furthermore, parts irrelevant to the explanation have been omitted from the drawings for clarity, and similar reference numerals have been assigned to similar parts throughout the specification.

Throughout the specification, when a part is said to “comprise” a certain component, it means that other components may be included unless specifically stated otherwise, rather than excluding other components.

In addition, the terms “ . . . unit” and “ . . . module” described in the specification refer to units that process at least one function or operation, and these can be implemented as hardware, software, or a combination of hardware and software.

Hereinafter, in one aspect, the facial blood flow-based stress measurement apparatus and method according to the embodiments of the present invention will be described in detail with reference to the drawings.

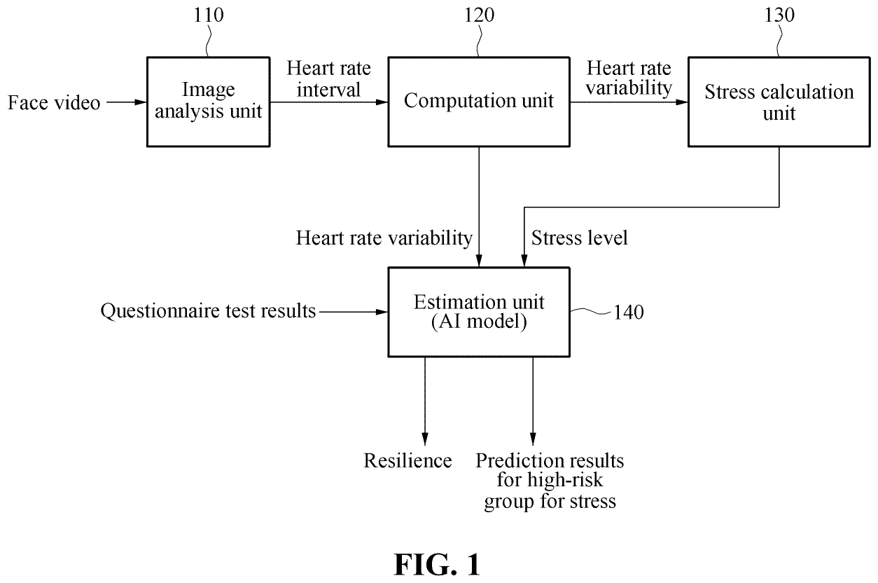

FIG. 1 illustrates a schematic configuration of a facial blood flow-based stress measurement apparatus according to the first embodiment of the present invention.

The facial blood flow-based stress measurement apparatus (hereinafter referred to as the stress measurement apparatus) according to the first embodiment of the present invention can be implemented as a stand-alone device independent of a network or as a server on a network.

In the case of a stand-alone device, the stress measurement apparatus can be a dedicated stress measurement device or a computing device such as a smartphone, tablet PC, laptop, or personal computer (PC), and the program installed and executed inside the device can measure stress independently without network access.

When implemented as a server on a network, the stress measurement apparatus is accessed by a web browser or client application installed on a computing device as a web-based or app-based platform, analyzes data received from the computing device, and provides stress measurement results to the computing device.

The stress measurement apparatus comprises an image analysis unit 110, a computation unit 120, a stress calculation unit 130, and an estimation unit 140.

The image analysis unit 110 analyzes a face video captured by a camera to measure heart rate intervals. The image analysis unit 110 can extract the location of arterial blood flow close to the facial skin from the face video and analyze the pixel data (RGB values) of the extracted location to measure heart rate intervals.

Generally, heart rate intervals are referred to as NN intervals (normal-to-normal intervals), but the name may vary slightly depending on the signal used.

When measuring heart rate intervals from an electrocardiogram (ECG) signal, the RR interval (R peak-to-R peak interval, RRI) represents the R-wave interval in the QRS waveform. When measuring heart rate intervals from a photoplethysmogram (PPG) signal, the PP interval (Peak-to-peak interval, PPI) represents the interval between peaks in the PPG waveform.

The computation unit 120 analyzes the heart rate interval measurement results output by the image analysis unit 110 to calculate heart rate variability.

In the embodiment of the present invention, heart rate variability (HRV) includes indicators analyzed in the time domain and indicators analyzed in the frequency domain for heart rate interval data collected over a certain period.

That is, when the image analysis unit 110 analyzes about 1 minute of face video to generate heart rate interval data, the computation unit 120 can calculate various indicators related to heart rate variability using the heart rate interval data for about 1 minute.

Time-domain analysis is a technique for analyzing changes in heart rate intervals over time and can calculate indicators such as beats per minute (BPM), SDNN (Standard deviation of all NN intervals), and RMSSD (Root mean square of successive differences between normal heartbeats).

Here, SDNN can be calculated using Mathematical Formula 1, and RMSSD can be calculated using Mathematical Formula 2.

$$SDNN = \sqrt{\frac{1}{N - 1} {\sum }_{i = 1}^{N} {[ Mean ( NN ) - {NN}_{i} ]}^{2}} \qquad [ Mathematical Formula 1 ]$$

$$RMSSD = \sqrt{\frac{1}{N - 2} {\sum }_{i = 2}^{N} {[ Mean ( NN ) - {NN}_{i - 1} ]}^{2}} \qquad [ Mathematical Formula 2 ]$$

Frequency-domain analysis is a technique for analyzing the ratio occupied in the frequency domain by frequency-converting heart rate interval data and can calculate indicators such as VLF (Very low frequency: 0.0033 to 0.04 Hz band), LF (Low frequency: 0.04 to 0.15 Hz band), HF (High frequency: 0.15 to 0.4 Hz band), and Total Power (0.0033 to 0.4 Hz band).

The stress calculation unit 130 calculates the stress level using various indicators related to heart rate variability calculated by the computation unit 120. The stress level can be classified into grades such as no stress, good, average, bad, and very depressed, and can be expressed as a stress score ranging from no stress (0) to very depressed (100).

The estimation unit 140 receives stress and internal vulnerability test result data, heart rate variability, and stress level as inputs, analyzes them through a machine learning-based model, and outputs prediction results for resilience and high-risk groups for stress.

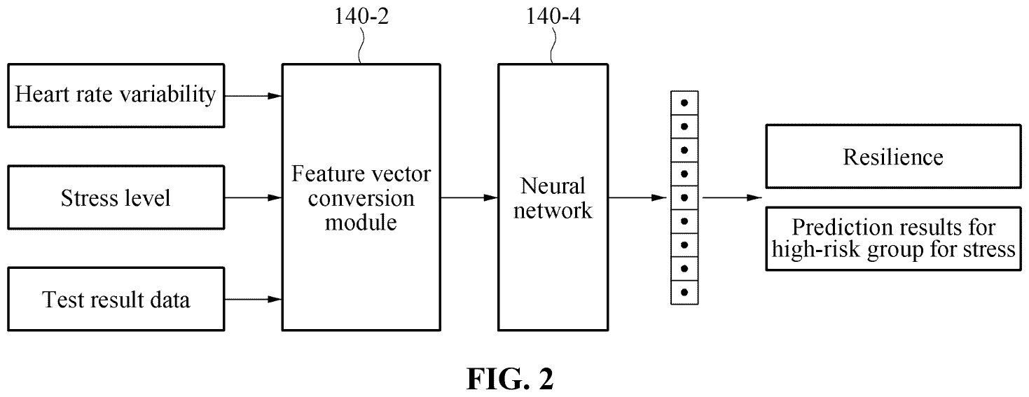

The stress and internal vulnerability test refers to a questionnaire-based assessment designed to evaluate a person's current stress level and their internal resilience against stress. FIG. 2 illustrates the internal configuration of the estimation unit 140 according to the first embodiment of the present invention.

Referring to FIG. 2, the estimation unit 140 comprises a feature vector conversion module 140-2 and a neural network 140-4.

The feature vector conversion module 140-2 receives heart rate variability, stress level, test result data, etc., and converts them into feature vectors applicable to the neural network (Deep Neural Network) 140-4. The feature vector is a value normalized and composed of N-dimensions for each piece of information.

The N-dimensional feature vector is input to the input layer of the neural network 140-4, passes through multiple hidden layers using weights and biases determined through the learning process and activation functions, and is output to the output layer. The value output to the output layer constitutes the stress analysis results, including the prediction results for resilience and high-risk groups for stress.

To implement the artificial intelligence-based neural network 140-4 according to the present invention, normalized values of heart rate variability, stress level, test result data, etc., are used as input data, and normalized values of prediction results for resilience and high-risk groups for stress are used as output data. The neural network 140-4 is trained through machine learning using training data.

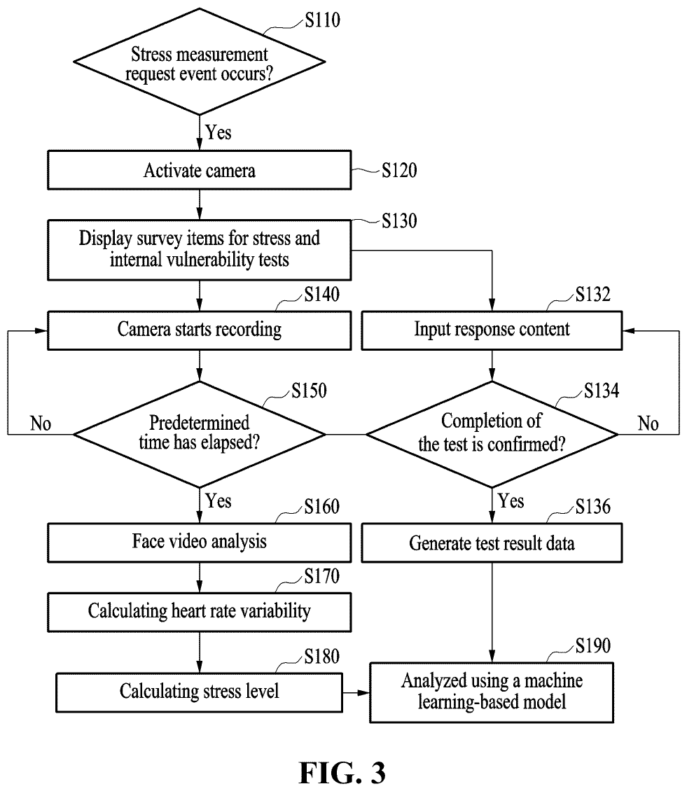

FIG. 3 illustrates the sequence process of the facial blood flow-based stress measurement method according to the first embodiment of the present invention.

Each step shown in FIG. 3 is performed by the facial blood flow-based stress measurement apparatus (e.g., smartphone, tablet PC, etc.) of the first embodiment. For convenience of explanation, the stress measurement apparatus, which is the subject of the operation, will be omitted.

Referring to FIG. 3, first, when a stress measurement request event occurs (S110), the camera is activated (S120), and at the same time, survey items for stress and internal vulnerability tests are displayed (S130).

Next, when the user presses the camera recording button, the camera starts recording (S140), and it is checked whether a predetermined time has elapsed (S150). The camera continues recording for a certain period. When the predetermined time has elapsed, the camera stops recording, and the face video obtained through the camera recording is analyzed (S160).

When heart rate interval data is generated through the analysis of the face video, the heart rate interval data is processed to calculate heart rate variability (S170).

When heart rate variability is calculated, the stress level is calculated using various indicators related to heart rate variability (S180).

Meanwhile, the user may take the stress and internal vulnerability tests by responding to the survey items displayed on the screen, while the camera is activated.

When the user responds to the survey items of the psychological stress assessment by clicking or touching, the response content is input (S132), the completion of the test is confirmed (S134), and the input of the test items continues until the test is completed. When the test is completed, test result data is generated (S136).

When the analysis of the facial video is completed, heart rate variability, stress levels, and psychological stress assessment results are further analyzed using a machine learning-based model (S190).

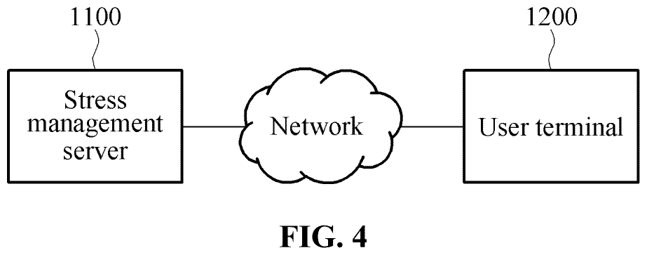

FIG. 4 illustrates the operation of a facial blood flow-based stress measurement apparatus according to the second embodiment of the present invention in connection with a network.

While the stress measurement apparatus of the first embodiment is an independent device from the network, the stress measurement apparatus of the second embodiment is a device inter-working with the network.

Referring to FIG. 4, a user terminal 1200 becomes the stress measurement apparatus of the second embodiment, and the user terminal 1200 inter-works with a stress management server 1100 on the network to receive stress measurement results from the stress management server 1100.

The stress management server 1100 is implemented on the network and has the same internal configuration as the stress measurement apparatus shown in FIG. 1.

That is, the stress management server 1100 receives the face video and test result data from the user terminal 1200, calculates the heart rate variability and stress level using the face video, analyzes the heart rate variability, stress level, and test result data using a machine learning-based model, outputs the psychological stress assessment results, which include resilience and high-risk group prediction results for stress, and provides the output values to the user terminal 1200.

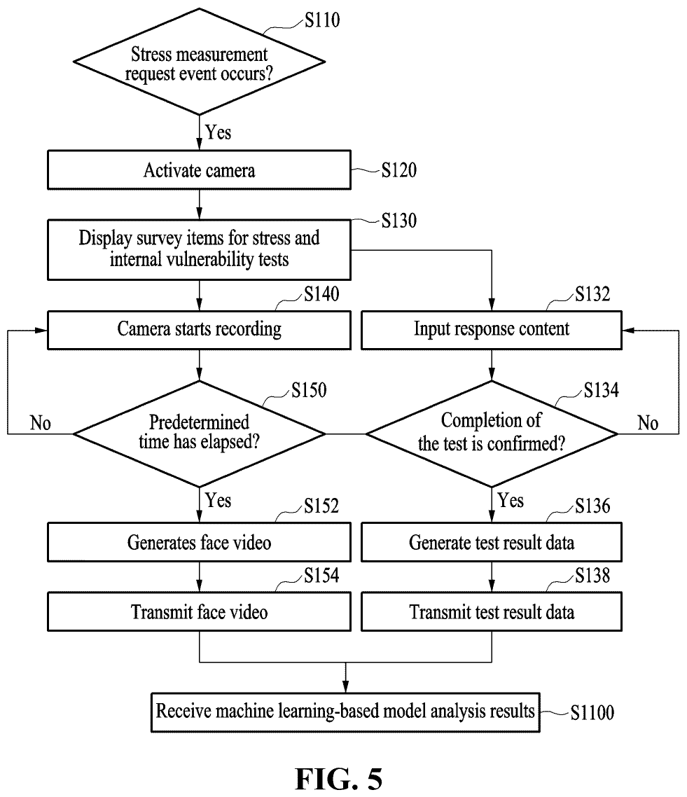

FIG. 5 illustrates the sequential process of the face recognition-based stress measurement method according to the second embodiment of the present invention.

Each step shown in FIG. 5 is performed by the user terminal 1200. For convenience of explanation, the user terminal 1200, which is the subject of the operation, will be omitted.

Referring to FIG. 5, first, when a stress measurement request event occurs (S110), the camera is activated (S120), and at the same time, survey items for stress and internal vulnerability tests are displayed (S130).

Next, when the user presses the camera capture button, the camera begins recording (S140), and it is determined whether a predetermined time has elapsed (S150). The camera continues to record for the set duration. Once the predetermined time has passed, the camera stops recording and generates a facial video file (S152).

When the face video file is generated, the face video file is compressed and transmitted to the stress management server 1100 (S154).

Meanwhile, the user can proceed with the psychological stress assessment by viewing the survey items for stress and internal vulnerability tests displayed on the screen along with the activation of the camera.

When the user responds to the survey items for the psychological stress assessment by clicking or touching, the response content is input (S132), the completion of the test is confirmed (S134), and the input of the test items continues until the test is completed. When the test is completed, test result data is generated (S136).

When the test result data is generated, the test result data is transmitted to the stress management server 1100 (S138).

When the face video and test result data are delivered to the stress management server 1100, the stress management server 1100 calculates the heart rate variability and stress level using the face video, analyzes the heart rate variability, stress level, and test result data using a machine learning-based model, and generates psychological stress assessment results.

Then, the user terminal 1200 receives the machine learning-based analysis results from the stress management server 1100 and outputs them to the user (S1100).

Hereinafter, in another aspect, a finger blood flow-based stress measurement apparatus and method according to an embodiment of the present invention will be described in detail with reference to the drawings.

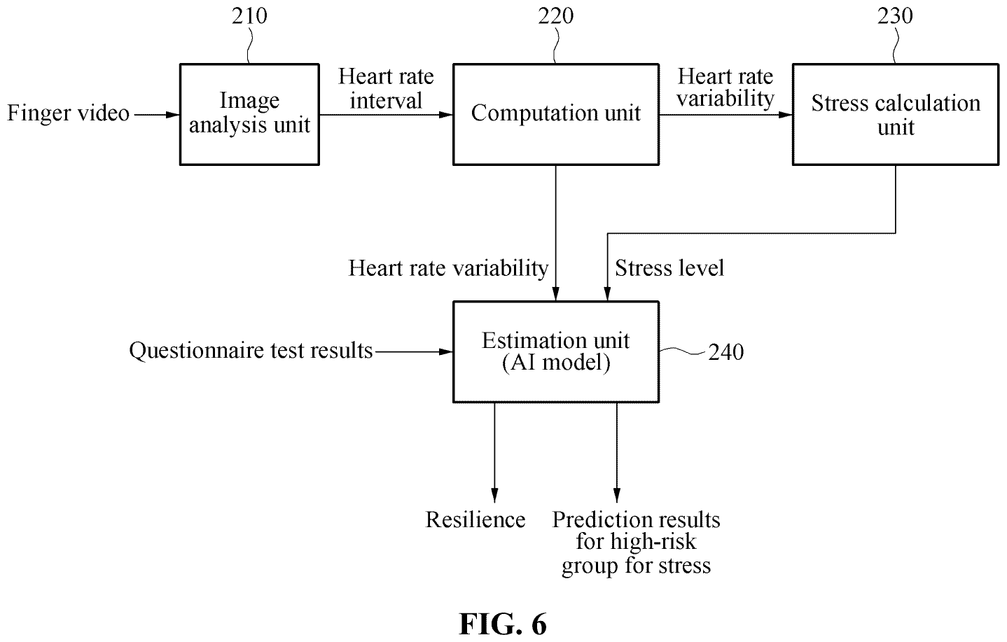

FIG. 6 illustrates a schematic configuration of a finger blood flow-based stress measurement apparatus according to the third embodiment of the present invention.

The finger blood flow-based stress measurement apparatus (hereinafter referred to as the stress measurement apparatus) according to the third embodiment of the present invention may be a stand-alone device independent of the network or may be implemented as a server on the network.

In the case of a stand-alone device, it may be a dedicated stress measurement device or a computing device such as a smartphone, tablet PC, laptop, or personal computer (PC), and the program installed and executed inside the device can measure stress independently without network access.

In the case of being implemented as a server on the network, the stress measurement apparatus is a web-based or app-based platform accessed by a web browser or client application installed on a computing device, analyzes data received from the computing device, and provides stress measurement results to the computing device.

The stress measurement apparatus comprises an image analysis unit 210, a computation unit 220, a stress calculation unit 230, and an estimation unit 240.

The image analysis unit 210 analyzes a finger video captured by a camera to measure heart rate (heartbeat) intervals. The image analysis unit 210 extracts a blood flow location close to the skin from the finger video and analyzes the pixel data (RGB values) of the extracted location to measure the heart rate intervals.

Generally, the heart rate interval is referred to as the NN interval (normal-to-normal interval, NN interval), but the name slightly varies depending on the signal used.

When measuring the heart rate interval from an electrocardiogram (ECG) signal, there is an RR interval (R peak-to-R peak interval, RRI) representing the R-wave interval in the QRS waveform. When measuring the heart rate interval from a photoplethysmogram (PPG) signal, there is a PP interval (Peak-to-peak interval, PPI) representing the interval between peaks in the PPG waveform.

The computation unit 220 analyzes the heart rate interval measurement results output by the image analysis unit 210 to calculate the heart rate variability.

In the embodiment of the present invention, heart rate variability (HRV) includes indicators analyzed in the time domain and indicators analyzed in the frequency domain for heart rate interval data collected over a certain period of time.

That is, when the image analysis unit 210 analyzes about 1 minute of finger video to generate heart rate interval data, the computation unit 220 can calculate various indicators related to heart rate variability using the heart rate interval data for about 1 minute.

Time-domain analysis is a technique for analyzing changes in heart rate intervals over time and can calculate indicators such as beats per minute (BPM), SDNN (Standard deviation of all NN intervals), and RMSSD (Root mean square of successive differences between normal heartbeats).

Here, SDNN can be calculated through Mathematical Formula 3, and RMSSD can be calculated through Mathematical Formula 4.

$$SDNN = \sqrt{\frac{1}{N - 1} {\sum }_{i = 1}^{N} {[ Mean ( NN ) - {NN}_{i} ]}^{2}} \qquad [ Mathematical Formula 3 ]$$

$$RMSSD = \sqrt{\frac{1}{N - 2} {\sum }_{i = 2}^{N} {[ Mean ( NN ) - {NN}_{i - 1} ]}^{2}} \qquad [ Mathematical Formula 4 ]$$

Frequency-domain analysis is a technique for analyzing the ratio occupied in the frequency domain by frequency-converting heart rate interval data and can calculate indicators such as VLF (Very low frequency: 0.0033 to 0.04 Hz band), LF (Low frequency: 0.04 to 0.15 Hz band), HF (High frequency: 0.15 to 0.4 Hz band), and Total Power (0.0033 to 0.4 Hz band).

The stress calculation unit 230 calculates the stress level using various indicators related to heart rate variability calculated by the computation unit 220. The stress level can be divided into grades such as no stress, good, average, bad, and very depressed, and can be expressed as a stress score ranging from no stress (0) to very depressed (100).

The estimation unit 240 receives the stress and internal vulnerability test result data, heart rate variability, and stress level, analyzes them through a machine learning-based model, and outputs resilience and high-risk group prediction results for stress.

The stress and internal vulnerability test refers to a survey test that can confirm the stress state and the internal resistance capability against stress through questionnaire items.

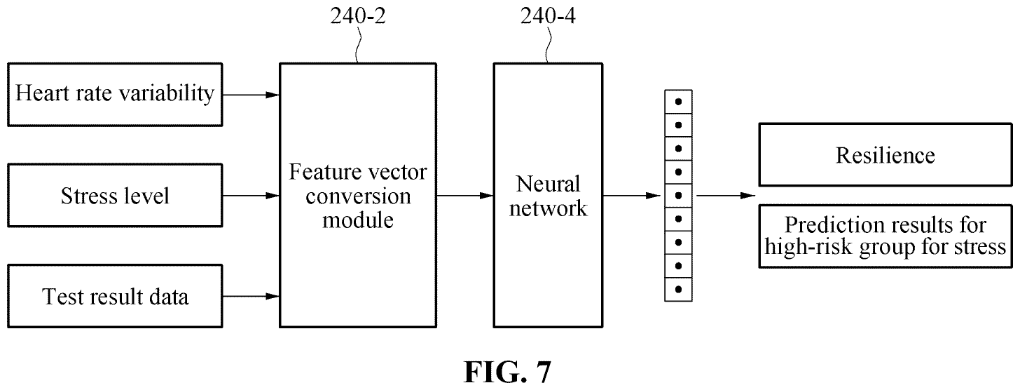

FIG. 7 illustrates the internal configuration of the estimation unit 240 according to the third embodiment of the present invention.

Referring to FIG. 7, the estimation unit 240 comprises a feature vector conversion module 240-2 and a neural network 240-4.

The feature vector conversion module 240-2 receives heart rate variability, stress level, test result data, and the like, and converts them into feature vectors applicable to the neural network (Deep Neural Network) 240-4. The feature vector is a value normalized and composed of N-dimensions.

The N-dimensional feature vector is input to the input layer of the neural network 240-4 and is output to the output layer through multiple hidden layers using weights and bias values determined through the learning process and an activation function. The value output to the output layer constitutes the stress analysis results, including resilience and high-risk group prediction results for stress.

To implement the artificial intelligence-based neural network 240-4 according to the present invention, the normalized values of heart rate variability, stress level, test result data, and the like are used as input data, and the normalized values of resilience and high-risk group prediction results for stress are used as output data. The neural network 240-4 is trained through machine learning using training data.

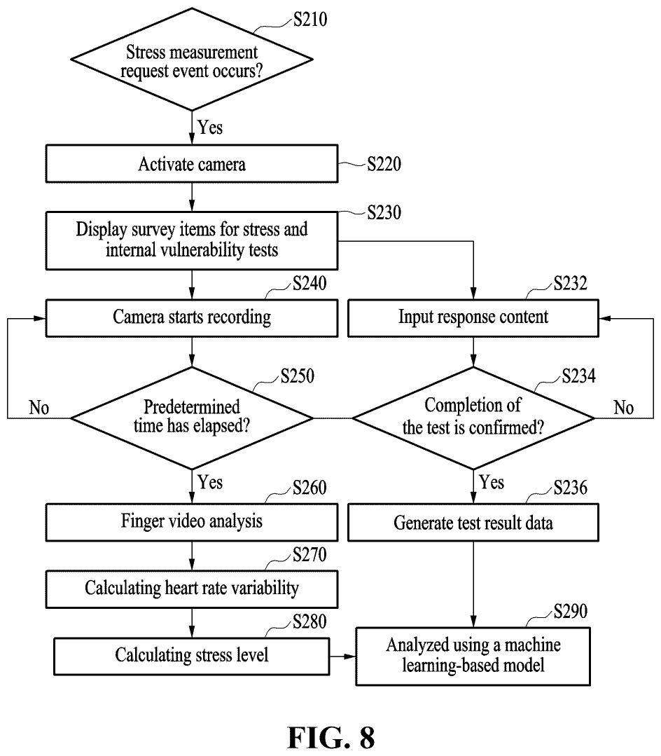

FIG. 8 illustrates the sequence process of the finger blood flow-based stress measurement method according to the third embodiment of the present invention.

Each step shown in FIG. 8 is performed in the finger blood flow-based stress measurement device (smartphone, tablet PC, etc.) of the third embodiment. For convenience of explanation, the stress measurement device, which is the subject of the operation, will be omitted.

Referring to FIG. 8, first, when a stress measurement request event occurs (S210), the camera is activated (S220), and at the same time, survey items for stress and internal vulnerability tests are output (S230).

Next, when the user presses the camera capture button, the camera begins recording (S240), and it is determined whether a predetermined amount of time has elapsed (S250). The camera continues recording during the specified time. Once the predetermined time has elapsed, the camera stops recording, and an analysis is performed on the finger video obtained through the recording process (S260).

When the analysis of the finger video generates heart rate interval data, the heart rate interval data is processed to calculate heart rate variability (S270).

When the heart rate variability is calculated, the stress level is calculated using various indicators related to the heart rate variability (S280).

Meanwhile, the user can proceed with the psychological stress assessment by viewing the survey items for stress and internal vulnerability tests displayed on the screen along with the activation of the camera.

In the case where the stress measurement device is a smartphone, the user holds the phone with one hand and shoots the finger of the other hand, which may make it difficult for the user to respond to the psychological stress assessment during recording.

Accordingly, the present invention can configure the psychological stress assessment screen so that the user can respond to the survey using the hand holding the phone.

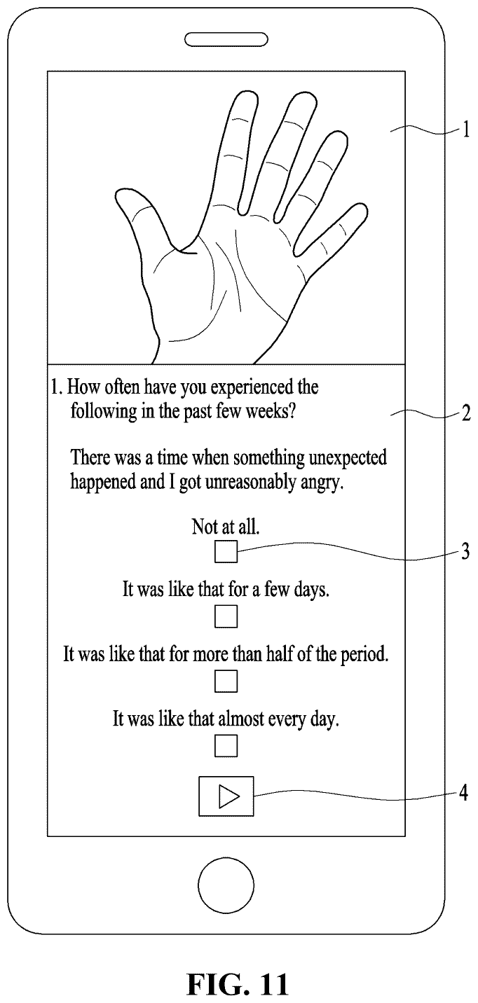

FIG. 11 illustrates the smartphone screen for finger blood flow-based stress measurement according to the present invention.

As shown in FIG. 11, the first area 1 of the screen displays the currently captured finger video, and the second area 2 displays the survey items for the psychological stress assessment.

Typically, when holding the phone with the right hand and recording the left-hand finger, the thumb of the right hand can move freely. In this case, the thumb is often located near the center of the second area 2 at the bottom of the screen, where it can stably touch. Therefore, the checkbox 3 for response input can be positioned near the center of the second area 2.

In the embodiment of the present invention, the checkbox 3 is illustrated as being located in the center as an example, but it is not limited to this. Various modifications are possible, such as positioning the response items for the survey (e.g., “Not at all,” “Several days,” etc.) in the center without a checkbox, and reversing or changing the color when touched.

In this way, the user can respond to the current survey using the thumb of the hand holding the phone while recording the finger video and continue to respond to the next survey in the same manner by touching the next button 4.

When the user responds to the psychological stress assessment items by clicking or touching, the response content is input (S232), the completion of the test is confirmed (S234), and the input of the test items continues until the test is completed. When the test is completed, test result data is generated (S236).

When the analysis of the finger video and the test are completed, the heart rate variability, stress level, psychological stress assessment result data, and the like are analyzed using a machine learning-based model (S290).

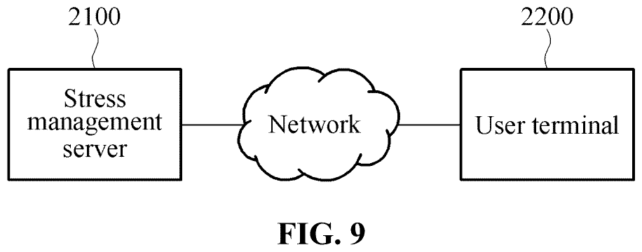

FIG. 9 illustrates the operation of the finger blood flow-based stress measurement device according to the fourth embodiment of the present invention in connection with a network.

While the stress measurement device of the third embodiment is an independent device from the network, the stress measurement device of the fourth embodiment is a network inter-working device.

Referring to FIG. 9, the user terminal 2200 becomes the stress measurement device of the fourth embodiment, and the user terminal 2200 interacts with the stress management server 2100 on the network to receive stress measurement results from the stress management server 2100.

The stress management server 2100 is implemented on the network and has the same internal configuration as the stress measurement device shown in FIG. 6.

That is, the stress management server 2100 receives the finger video and test result data from the user terminal 2200, calculates heart rate variability and stress level using the finger video, and analyzes the heart rate variability, stress level, and test result data using a machine learning-based model to output resilience and high-risk group prediction result information for stress as the psychological stress assessment result. This output value is provided to the user terminal 2200.

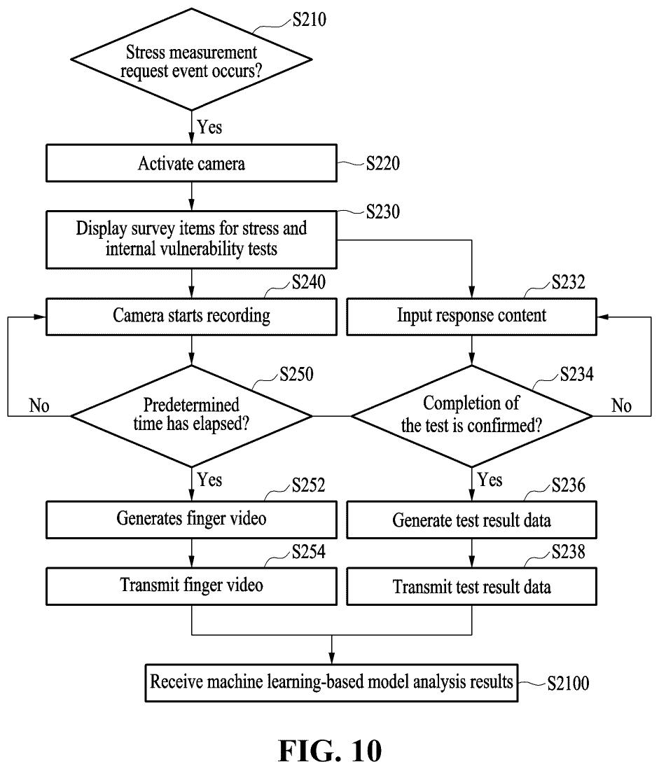

FIG. 10 illustrates the sequence process of the finger blood flow-based stress measurement method according to the fourth embodiment of the present invention.

Each step shown in FIG. 10 is performed in the user terminal 2200. For convenience of explanation, the user terminal 2200, which is the subject of the operation, will be omitted.

Referring to FIG. 10, first, when a stress measurement request event occurs (S210), the camera is activated (S220), and at the same time, survey items for stress and internal vulnerability tests are output (S230).

Next, when the user presses the camera recording button, the camera starts recording (S240), and it is checked whether a predetermined time has elapsed (S250). The camera continues recording for a certain period of time. When the predetermined time has elapsed, the camera stops recording and generates a finger video file (S252).

When the finger video file is generated, the finger video file is compressed and transmitted to the stress management server 2100 (S254).

Meanwhile, the user can proceed with the psychological stress assessment by viewing the survey items for stress and internal vulnerability tests displayed on the screen shown in FIG. 11 along with the activation of the camera.

When the user responds to the psychological stress assessment items by clicking or touching, the response content is input (S232), the completion of the test is confirmed (S234), and the input of the test items continues until the test is completed. When the test is completed, test result data is generated (S236).

When the test result data is generated, the test result data is transmitted to the stress management server 2100 (S238).

When the finger video and test result data are delivered to the stress management server 2100, the stress management server 2100 calculates heart rate variability and stress level using the finger video, and analyzes the heart rate variability, stress level, and test result data using a machine learning-based model to generate psychological stress assessment results.

Then, the user terminal 2200 receives the machine learning-based analysis results from the stress management server 2100 and outputs them to the user (S2100).

Hereinafter, in another aspect, the user-customized stress care device and method according to the embodiment of the present invention will be described in detail with reference to the drawings.

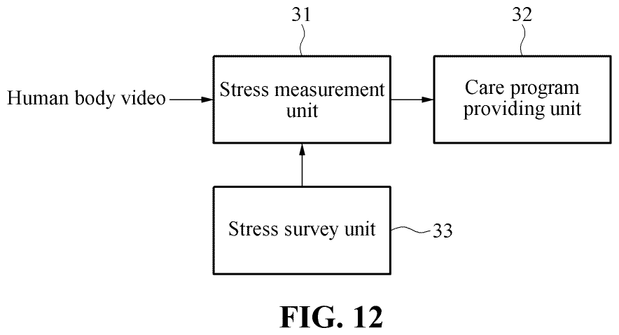

FIG. 12 illustrates a schematic configuration of the user-customized stress care device according to the fifth embodiment of the present invention.

The user-customized stress care device according to the fifth embodiment of the present invention (hereinafter referred to as the stress care device) may be implemented as a stand-alone device independent of the network or as a server on the network (inter-working device).

In the case of a stand-alone device, it may be a dedicated stress care device or a computing device such as a smartphone, tablet PC, laptop, or personal computer (PC). The program installed and executed inside the device can measure stress independently without network access and provide a stress management program accordingly.

When implemented as a server on a network, the stress care device is accessed by a web browser or client application installed on a computing device as a web-based or app-based platform, analyzes data received from the computing device, and provides the stress measurement result and the corresponding stress management program to the computing device.

The stress care device comprises a stress measurement unit 31, a care program providing unit 32, a stress survey unit 33, and the like.

The stress measurement unit 31 analyzes a human body video captured by a camera and stress and internal vulnerability test result data generated by the stress survey unit 33 to output a stress measurement result. Here, the human body video may be a face or finger video, and the stress measurement result may include a prediction result of resilience and a high-risk group for stress.

The care program providing unit 32 executes a stress management program according to the stress measurement result. The stress management program consists of a plurality of care processes, and the care program providing unit 32 can sequentially execute and provide each care process to the user.

The stress survey unit 33 can provide various survey items for stress and internal vulnerability tests to the user. The stress survey unit 33 can provide survey items consisting of multiple-choice and subjective questions. The stress survey unit 33 receives the response content when the user responds to the survey items and generates test result data.

For example, the stress survey unit 33 can generate test result data by analyzing key keywords or content from the response content provided by the user.

During the execution of the stress management program, the stress measurement unit 31 outputs a stress measurement result whenever each care process is completed, and when the entire stress management program is completed, it can calculate a change value between the initial stress measurement result and the final stress measurement result. This change value indicates how much the stress state has improved through daily stress care.

The care program providing unit 32 can select and provide the stress management program to be conducted the next day based on the daily change value of the stress state.

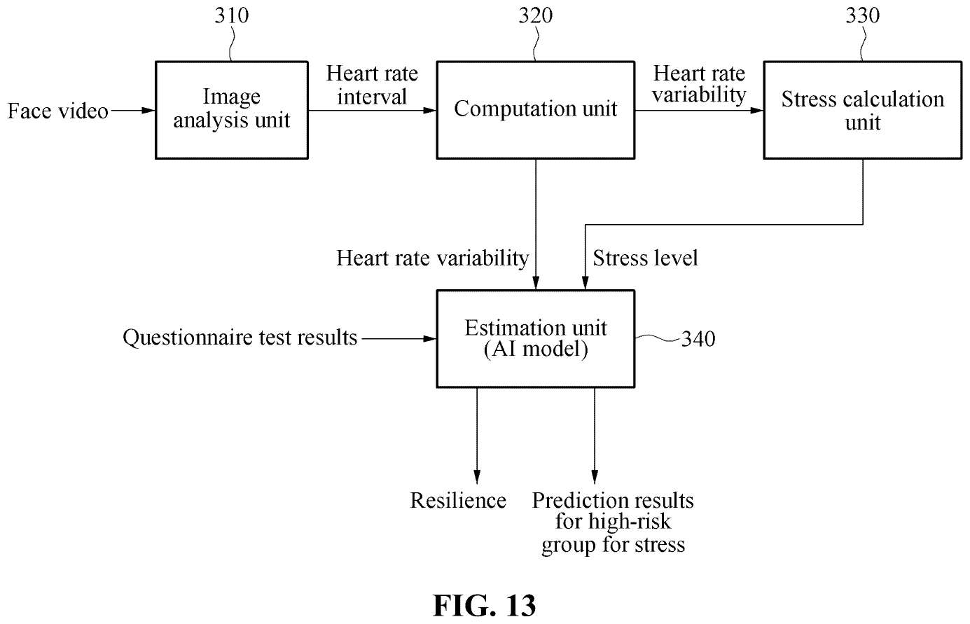

FIG. 13 illustrates the internal configuration of the stress measurement unit of the user-customized stress care device according to the fifth embodiment of the present invention.

Referring to FIG. 13, the stress measurement unit 31 comprises an image analysis unit 310, a computation unit 320, a stress calculation unit 330, an estimation unit 340, and the like. In FIG. 13, the measurement of the stress state using a face video is described as an example. The image analysis unit 310 analyzes a face (facial) video captured by a camera to measure heart rate (heartbeat) intervals. The image analysis unit 310 extracts a blood flow location close to the facial skin from the face video and analyzes the pixel data (RGB values) of the extracted location to measure heart rate intervals.

Generally, heart rate intervals are referred to as NN intervals (normal-to-normal intervals, NN intervals), but the name slightly varies depending on the signal used.

When measuring heart rate intervals from an electrocardiogram (ECG) signal, there is an RR interval (R peak-to-R peak interval, RRI) representing the R-wave interval in the QRS waveform. When measuring heart rate intervals from a photoplethysmogram (PPG) signal, there is a PP interval (Peak-to-peak interval, PPI) representing the interval between peaks in the PPG waveform.

The computation unit 320 analyzes the heart rate interval measurement result output by the image analysis unit 310 to calculate heart rate variability.

In the embodiment of the present invention, heart rate variability (HRV: Heart Rate Variability) includes indicators analyzed in the time domain and indicators analyzed in the frequency domain for heart rate interval data collected over a certain period.

That is, when the image analysis unit 310 analyzes about one minute of face video to generate heart rate interval data, the computation unit 320 can calculate various indicators related to heart rate variability using the heart rate interval data for about one minute.

Time-domain analysis is a technique for analyzing changes in heart rate intervals over time and can calculate indicators such as beats per minute (BPM: Beat per minute), SDNN (Standard deviation of all NN intervals), and RMSSD (Root mean square of successive differences between normal heartbeats).

Here, SDNN can be calculated through Mathematical Formula 5, and RMSSD can be calculated through Mathematical Formula 6.

$$SDNN = \sqrt{\frac{1}{N - 1} {\sum }_{i = 1}^{N} {[ Mean ( NN ) - {NN}_{i} ]}^{2}} \qquad [ Mathematical Formula 5 ]$$

$$RMSSD = \sqrt{\frac{1}{N - 2} {\sum }_{i = 2}^{N} {[ Mean ( NN ) - {NN}_{i - 1} ]}^{2}} \qquad [ Mathematical Formula 6 ]$$

Frequency-domain analysis is a technique for frequency-converting heart rate interval data and analyzing the ratios occupied in the frequency domain. It can calculate indicators such as VLF (Very low frequency: 0.0033 to 0.04 Hz band), LF (Low frequency: 0.04 to 0.15 Hz band), HF (High frequency: 0.15 to 0.4 Hz band), and Total Power (0.0033 to 0.4 Hz band).

The stress calculation unit 330 calculates the stress level using various indicators related to heart rate variability calculated by the computation unit 320. The stress level can be graded as no stress, good, average, bad, very depressed, etc., and can be expressed as a stress score ranging from no stress (0) to very depressed (100).

The estimation unit 340 receives stress and internal vulnerability test result data, heart rate variability, and stress level, analyzes them through a machine learning-based model, and outputs a prediction result of resilience and a high-risk group for stress.

The stress and internal vulnerability test refers to a survey test that identifies the stress state and the state of internal resistance to stress through questionnaire items.

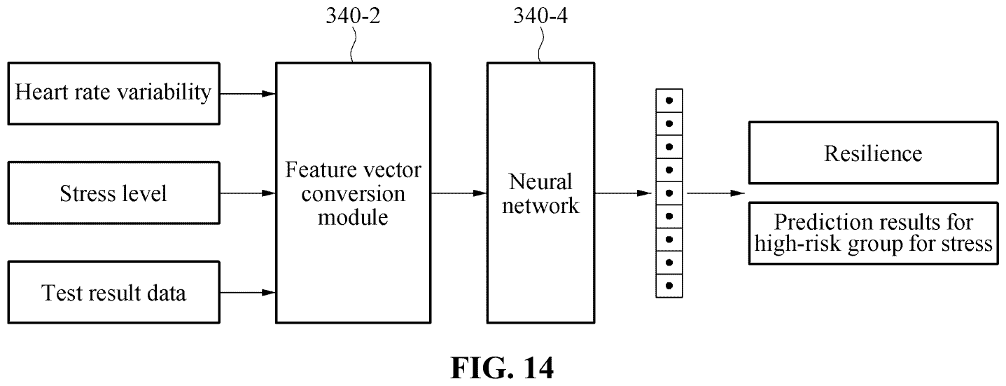

FIG. 14 illustrates the internal configuration of the estimation unit 340 according to the fifth embodiment of the present invention.

Referring to FIG. 14, the estimation unit 340 consists of a feature vector conversion module 340-2 and a neural network 340-4.

The feature vector conversion module 340-2 receives heart rate variability, stress level, test result data, and the like, and converts them into feature vectors applicable to the neural network (Deep Neural Network) 340-4. The feature vector is a value normalized and composed of N-dimensions for each piece of information.

The N-dimensional feature vector is input to the input layer of the neural network 340-4, and through the learning process, it passes through multiple hidden layers using weights and biases determined during training and activation functions, and is output to the output layer. The value output to the output layer constitutes the stress analysis result, which includes the prediction result of resilience and a high-risk group for stress.

To implement the artificial intelligence-based neural network 340-4 according to the present invention, normalized values of heart rate variability, stress level, test result data, and the like are used as input data, and normalized values of the prediction result of resilience and a high-risk group for stress are used as output data. The neural network 340-4 is trained through machine learning using training data.

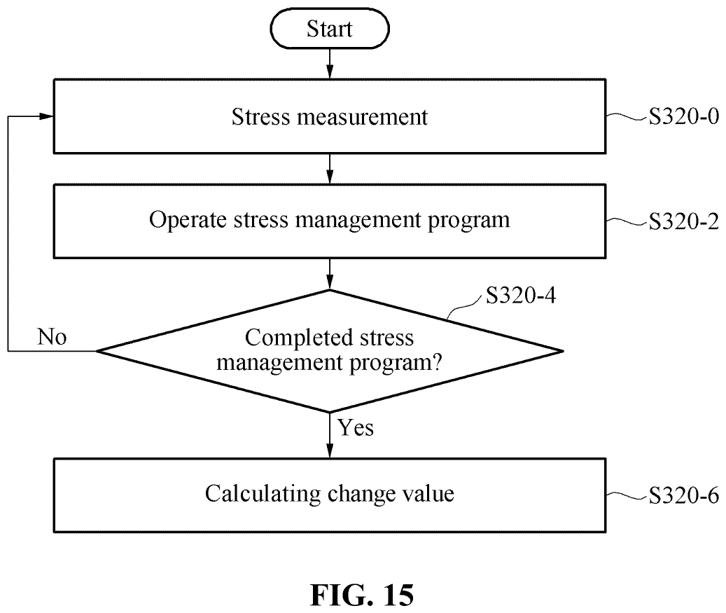

FIG. 15 illustrates the sequence process of the user-customized stress care method according to the fifth embodiment of the present invention.

Each step shown in FIG. 15 is performed by the user-customized stress care device (smartphone, tablet PC, etc.) of the fifth embodiment. For convenience of explanation, the stress care device, which is the subject of the operation, will be omitted.

Referring to FIG. 15, first, a stress measurement result, which is a prediction result of resilience and a high-risk group for stress, is output using stress and internal vulnerability test result data and a human body video (S320-0).

When the stress measurement result is output, a stress management program according to the stress measurement result is operated (S320-2).

Since the stress management program includes a plurality of care processes, it is checked whether each care process has been executed and the stress management program has been completed (S320-4).

If the stress management program has not yet been completed, the stress measurement step (S320-0) and the stress management program operation step (S320-2) are repeated whenever each care process is completed. When all care processes are completed, a change value between the initial stress measurement result and the final stress measurement result is calculated (S320-6). This change value is stored in the memory of the stress care device and serves as a criterion for selecting the program when the next healing program is executed.

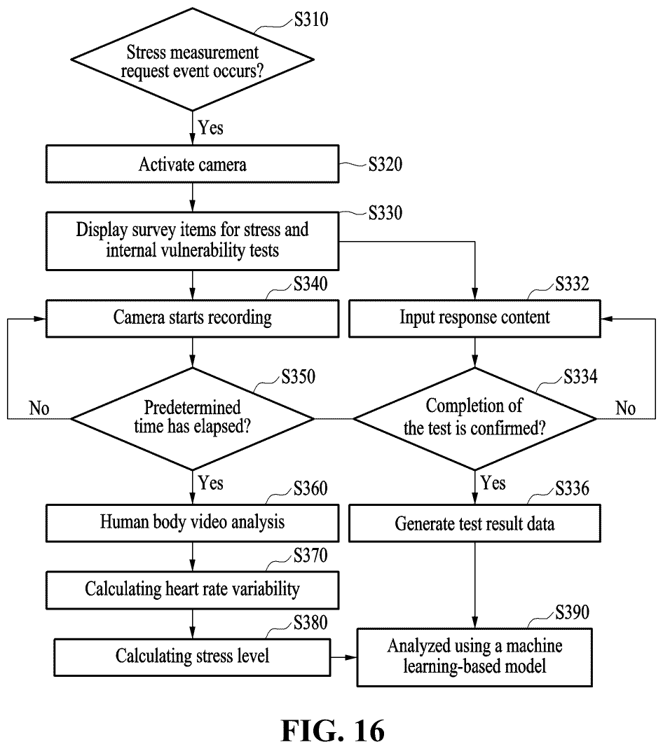

FIG. 16 is a flowchart specifically illustrating the stress measurement step (S320-0) in the user-customized stress care method according to the fifth embodiment of the present invention.

Referring to FIG. 16, first, when a stress measurement request event occurs (S310), the camera is activated (S320), and at the same time, survey items for stress and internal vulnerability tests are displayed (S330).

Next, when the user presses the camera recording button, the camera starts recording (S340), and it is checked whether a certain amount of time has elapsed (S350), and the camera continues recording for a predetermined time. When the predetermined time elapses, the camera recording is terminated, and an analysis of the human body video obtained through the camera recording is performed (S360).

When heart rate interval data is generated through the analysis of the human body video, the heart rate interval data is processed to calculate heart rate variability (S370).

When the heart rate variability is calculated, the stress level is calculated using various indicators related to the heart rate variability (S380).

Meanwhile, the user can proceed with the psychological stress assessment by viewing the survey items for stress and internal vulnerability tests displayed on the screen along with the activation of the camera.

When the user responds to the survey items for the psychological stress assessment by clicking or touching, the response content is input (S332), the completion of the test is confirmed (S334), and the input of the survey items continues until the test is completed. Once the test is completed, test result data is generated (S336).

When the analysis of the human body video and the test are completed, the heart rate variability, stress level, and psychological stress assessment result data are analyzed using a machine learning-based model (S390).

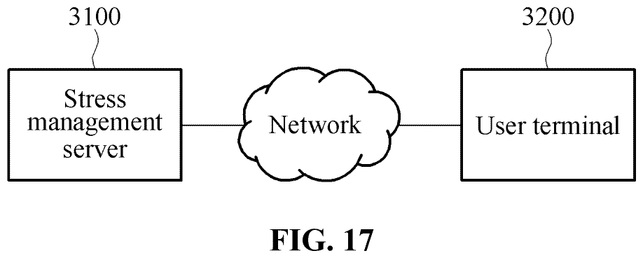

FIG. 17 illustrates the operation of the user-customized stress care device according to the sixth embodiment of the present invention in connection with a network.

While the stress care device of the fifth embodiment is a device independent of the network, the stress care device of the sixth embodiment is a device inter-working with the network.

Referring to FIG. 17, the user terminal 3200 becomes the stress care device of the sixth embodiment, and the user terminal 3200 interworks with the stress management server 3100 on the network to receive stress measurement results and stress management programs from the stress management server 3100.

The stress management server 3100 is implemented on the network and has the same internal configuration as the stress care device shown in FIG. 12.

That is, the stress management server 3100 receives the human body video and test result data from the user terminal 3200, calculates heart rate variability and stress level using the human body video, and analyzes the heart rate variability, stress level, and test result data using a machine learning-based model to output the stress measurement result, which is the prediction result of resilience and a high-risk group for stress, and provides this output value to the user terminal 3200.

Additionally, the stress management server 3100 selects a stress management program according to the psychological stress assessment result and provides it to the user terminal 3200.

The user terminal 3200 captures a human body video at the end of each care process of the stress management program and transmits it to the stress management server 3100, and the stress management server 3100 analyzes the human body video and transmits the stress measurement result to the user terminal 3200.

When the stress management program is fully completed, the stress management server 3100 stores the stress measurement result data from the beginning to the end and the change value between the first and last stress measurement results in an internal database.

The sequence of the user-customized stress care method according to the sixth embodiment of the present invention is almost identical to the flowchart of FIG. 15.

However, while each step in FIG. 15 is performed by the stress care device independent of the network in the fifth embodiment, in the sixth embodiment, each step is performed by the stress management server on the network, and the results of the performance are displayed on the user terminal 3200 connected to the network.

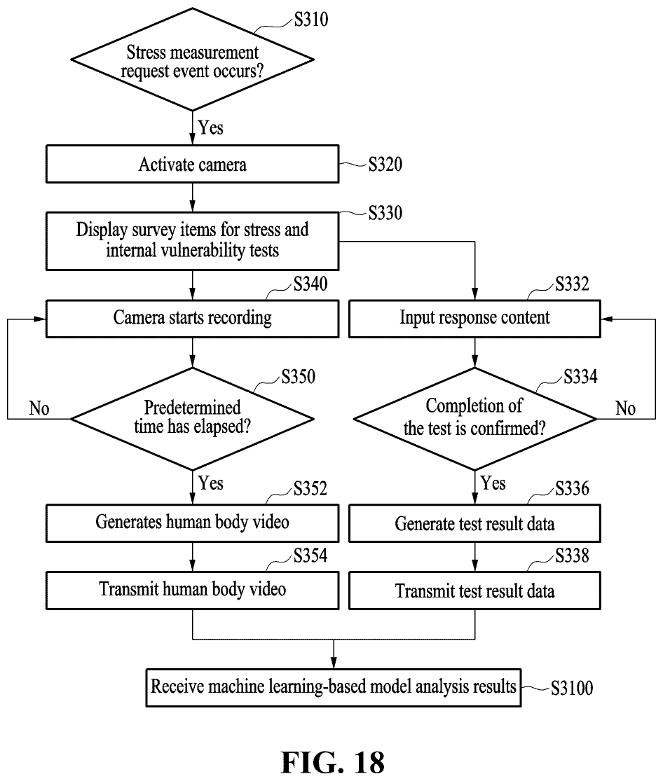

FIG. 18 illustrates the stress measurement process in the user-customized stress care method according to the sixth embodiment of the present invention.

Each step shown in FIG. 18 is performed on the user terminal 3200. For convenience of explanation, the user terminal 3200, which is the subject of the operation, will be omitted.

Referring to FIG. 18, first, when a stress measurement request event occurs (S310), the camera is activated (S320), and at the same time, survey items for stress and internal vulnerability tests are displayed (S330).

Next, when the user presses the camera recording button, the camera starts recording (S340), and it is checked whether a certain amount of time has elapsed (S350), and the camera continues recording for a predetermined time. When the predetermined time elapses, the camera recording is terminated, and a human body video file is generated (S352).

When the human body video file is generated, the human body video file is compressed and transmitted to the stress management server 3100 (S354).

Meanwhile, the user can proceed with the psychological stress assessment by viewing the survey items for stress and internal vulnerability tests displayed on the screen along with the activation of the camera.

When the user responds to the survey items for the psychological stress assessment by clicking or touching, the response content is input (S332), the completion of the test is confirmed (S334), and the input of the survey items continues until the test is completed. Once the test is completed, test result data is generated (S336).

When the test result data is generated, the test result data is transmitted to the stress management server 3100 (S338).

When the human body video and test result data are delivered to the stress management server 3100, the stress management server 3100 calculates heart rate variability and stress level using the human body video, and analyzes the heart rate variability, stress level, and test result data using a machine learning-based model to generate the psychological stress assessment result.

Then, the user terminal 3200 receives the machine learning-based analysis result from the stress management server 3100 and outputs it to the user (S3100).

Subsequently, although not shown in FIG. 18, the user terminal 3200 receives a stress management program according to the analysis result from the stress management server 3100.

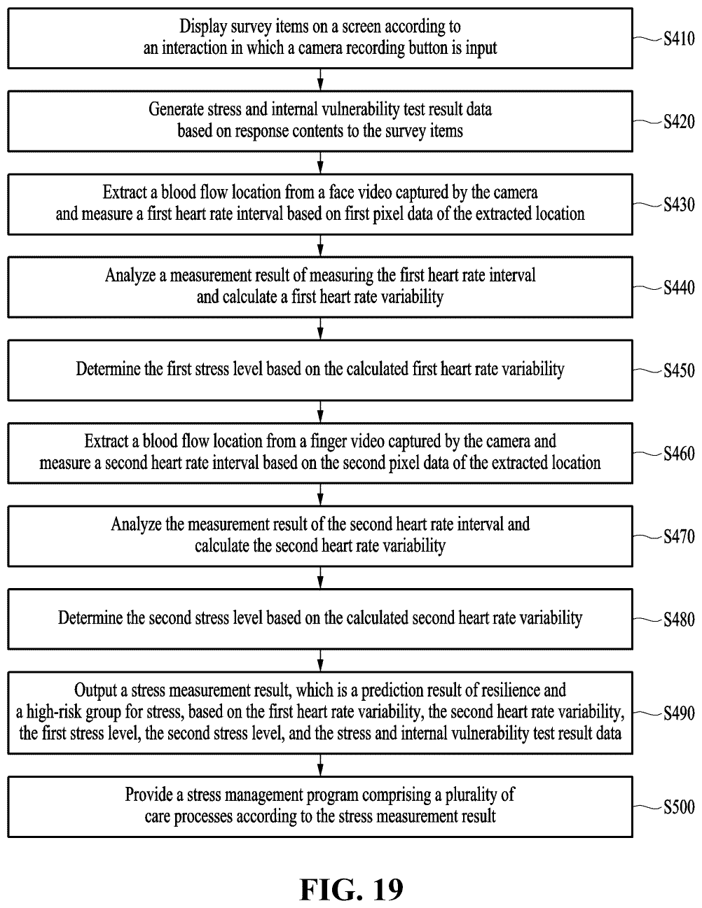

FIG. 19 illustrates another sequence of the stress measurement method according to some embodiments of the present invention. FIG. 19 explains a method of measuring stress and providing a stress management program based on the previously described face and finger videos. In FIG. 19, the psychological stress assessment measurement device may comprise an image analysis unit 410, a computation unit 420, a stress calculation unit 430, an estimation unit 440, a care program providing unit 450, and a stress survey unit 460.

Referring to FIG. 19, the stress survey unit 460 may display survey items on a screen according to an interaction in which a camera recording button is input (S410). The stress survey unit 460 may generate stress and internal vulnerability test result data based on response contents to the survey items (S420).

Specifically, the stress measurement device may start capturing a user using the camera according to an interaction selecting the camera recording button from the user. The stress survey unit 460 may display the survey items on the screen while the user is being captured. Then, the stress measurement device may terminate the capturing of the user when a preset time related to the recording time has elapsed.

For example, the stress measurement device may terminate the capturing of the user when it is determined that 1 minute has elapsed since the interaction occurred.

The image analysis unit 410 may extract a blood flow location from a face video captured by the camera and measure a first heart rate interval based on first pixel data of the extracted location (S430). The computation unit 420 may analyze a measurement result of measuring the first heart rate interval and calculate a first heart rate variability (S440).

The first heart rate variability may comprise a time domain index analyzed in a time domain and a frequency domain index analyzed in a frequency domain for heart rate interval data collected for a predetermined time. Here, the time domain index may comprise a standard deviation of normal-to-normal intervals (SDNN) and a root mean square of successive differences between normal heartbeats (RMSSD).

The standard deviation of heart rate intervals may be determined based on the mathematical formula described earlier. Here, and are natural numbers, and may be the first heart rate interval. The root mean square of successive differences of heart rate intervals may be determined based on the mathematical formula described earlier.

The frequency domain index may represent the ratios occupied by VLF (Very low frequency: 0.0033 to 0.04 Hz band), LF (Low frequency: 0.04 to 0.15 Hz band), and HF (High frequency: 0.15 to 0.4 Hz band) in a frequency domain by frequency-converting heart rate interval data.

The stress calculation unit 430 may determine the first stress level based on the calculated first heart rate variability (S450).

The image analysis unit 410 may extract a blood flow location from a finger video captured by the camera and measure a second heart rate interval based on the second pixel data of the extracted location (S460). The computation unit 420 may analyze the measurement result of the second heart rate interval and calculate the second heart rate variability (S470). Steps S460 to S480 are described after step S450 for convenience of explanation but may be executed before step S430.

The second heart rate variability may include a time domain index analyzed in a time domain and a frequency domain index analyzed in a frequency domain for heart rate interval data collected for a predetermined time. Here, the time domain index may include the standard deviation of heart rate intervals (SDNN) and the root mean square of successive differences (RMSSD) between heart rate intervals.

The standard deviation of heart rate intervals may be determined based on the mathematical formula described earlier. Here, and are natural numbers, and may be the second heart rate interval. The root mean square of successive differences of heart rate intervals may be determined based on the mathematical formula described earlier.

The frequency domain index may represent the ratios occupied by VLF (Very low frequency: 0.0033 to 0.04 Hz band), LF (Low frequency: 0.04 to 0.15 Hz band), and HF (High frequency: 0.15 to 0.4 Hz band) in a frequency domain by frequency-converting heart rate interval data.

The stress calculation unit 430 may determine the second stress level based on the calculated second heart rate variability (S480).

The estimation unit 440 may output a stress measurement result, which is a prediction result of resilience and a high-risk group for stress, based on the first heart rate variability, the second heart rate variability, the first stress level, the second stress level, and the stress and internal vulnerability test result data (S490).

Specifically, the estimation unit 440 may determine the stress measurement result by analyzing the first heart rate variability, the second heart rate variability, the first stress level, the second stress level, and the stress and internal vulnerability test result data through a machine learning-based model.

The care program providing unit 450 may provide a stress management program comprising a plurality of care processes according to the stress measurement result (S500).

Specifically, the care program providing unit 450 may determine a care plan in which the plurality of care processes are provided. The care plan may include first plan information for determining at least some care processes among the plurality of care processes and second plan information for determining an order in which the at least some care processes are provided. In other words, the care program providing unit 450 may determine at least some care processes among the plurality of care processes to be provided to the user. The care program providing unit 450 may determine the order in which the at least some care processes are provided. The plurality of care processes may include, for example, a meditation process, a music appreciation process, a video appreciation process, a singing process, a gaming process, a chatbot conversation process, a process for enjoying food, a process for making phone calls with people around, or a process for consulting with a counselor. The plurality of care processes may include user-customized care processes generated using test result data. For example, the stress survey unit 33 may receive response contents related to situations the user wants to overcome through stress and internal vulnerability tests. The care program providing unit 450 may generate user-customized care processes based on the input response contents. Then, the care program providing unit 450 may determine a care plan comprising the user-customized care processes.

The care program providing unit 450 may provide at least some care processes according to the determined care plan. The care program providing unit 450 may output a sub-stress measurement result according to each of the at least some care processes when each of the at least some care processes is terminated. For example, the care program providing unit 450 may provide a first care process according to the result of the care plan. When the first care process is terminated, the care program providing unit 450 may output a sub-stress measurement result according to the first care process. The care program providing unit 450 may provide a second care process according to the result of the care plan. When the second care process is terminated, the care program providing unit 450 may output a sub-stress measurement result according to the second care process. The care program providing unit 450 may provide a third care process according to the result of the care plan. When the third care process is terminated, the care program providing unit 450 may output a sub-stress measurement result according to the third care process.

The care program providing unit 450 may calculate a change value between a first sub-stress measurement result measured first and a second sub-stress measurement result measured last when all of the at least some care processes are provided. Then, the care program providing unit 450 may output the calculated change value. The change value may represent the degree to which the user's stress has decreased or increased, for example, as a percentage. According to one embodiment, the stress measurement device may start capturing the user using the camera in conjunction with the operation of providing at least some care processes by the care program providing unit 450. Then, the stress measurement device may terminate capturing the user when all of the at least some care processes are provided. Accordingly, the user may conveniently receive the care program without performing interactions such as inputting a camera recording button.

According to one embodiment, the care program providing unit 450 may determine whether a past change value determined within a preset previous time period from the current point in time exists in determining a care plan for the plurality of care processes.

For example, the current point in time may be assumed to be 6:55:00 PM KST on Mar. 16, 2025. In this case, the preset previous time period may be the 24 hours prior to 12:00:00 AM KST on Mar. 16, 2025. In other words, the preset previous time period may be from 12:00:00 AM KST on Mar. 15, 2025, to 11:59:59 PM KST on Mar. 15, 2025. In other words, the preset previous time period may be the previous day from the current point in time.

When it is determined that a past change value exists, the care program providing unit 450 may determine a care plan for the plurality of care processes based on the past change value.

Specifically, the care program providing unit 450 may determine a care plan for the plurality of care processes based on a past care provision history applied to calculate the past change value when the past change value is equal to or greater than a predetermined threshold.

For example, the care program providing unit 450 may determine a past care provision history that was used for calculating a past change value. The care program providing unit 450 may determine a care plan based on the determined past care provision history. The care program providing unit 450 may determine a current care plan such that at least some care processes are determined similarly to the past care provision history.

The care program providing unit 450 may determine a care plan for a plurality of care processes without using the past change value when the past change value is less than a predetermined threshold.

According to one embodiment, the care program providing unit 450 may determine the number of times a specific care combination has been used based on identification information related to the user. The specific care combination may be understood as being the same as or similar to the past care provision history. The specific care combination may include first plan information in which at least some care processes among the plurality of care processes appearing in the past care provision history are selected, and second plan information in which the order in which at least some care processes are provided is selected. The identification information may include the user's ID (identifier) or phone number. The number of times the past combination has been used may be understood as the number of times the plurality of care processes were provided as the past combination. The care program providing unit 450 may determine a combination of a plurality of care processes that is similar to the past combination by at least a predetermined threshold when the number of times the past combination has been used is less than the predetermined threshold.

For example, the predetermined threshold may be assumed to be three times. When the number of times a specific care combination has been used is less than three times, the care program providing unit 450 may decide to use the specific care combination. The care program providing unit 450 may determine a care plan for a plurality of care processes that is similar to the specific care combination by at least the predetermined threshold. The care program providing unit 450 may determine at least some care processes used in the specific care combination. The care program providing unit 450 may determine the order in which at least some care processes were provided in the specific care combination.

The care program providing unit 450 may determine a care plan such that the determined at least some care processes and the order in which at least some care processes are provided are similar to the specific care combination by at least the predetermined threshold. Even if the user's stress reduction is significant in a stress management program provided according to the specific care combination, if the same program is provided repeatedly, the user may lose interest or the stress may not be reduced. Therefore, the care program providing unit 450 may determine a care plan based on the number of times the specific care combination has been used. If the number of times the specific care combination has been used is equal to or greater than the predetermined threshold, the care program providing unit 450 may determine a care plan in which the care processes provided in the specific care combination differ from the care processes provided by at least the predetermined threshold, and the order of the provided care processes also differs. Accordingly, care processes different from those provided in the past are provided, and the user may feel newly interested.

The embodiments of the present invention described above are not implemented only through devices and methods but may also be implemented through a program that realizes the functions corresponding to the configuration of the embodiments of the present invention or a recording medium on which the program is recorded.

The above description is merely illustrative of the present invention, and various modifications may be made without departing from the technical spirit of the present invention by those skilled in the art to which the present invention pertains.

Therefore, the embodiments disclosed in the specification of the present invention are not intended to limit the present invention. The scope of the present invention should be interpreted based on the following claims, and all technologies within the equivalent scope thereof should be interpreted as being included in the scope of the present invention.

### EXPLANATION OF REFERENCE NUMERALS

110: Image analysis unit 120: Computation unit
130: Stress calculation unit 140: Estimation unit
1100: Stress management server 1200: User terminal
210: Image analysis unit 220: Computation unit
230: Stress calculation unit 240: Estimation unit
2100: Stress management server 2200: User terminal
31: Stress measurement unit 32: Care program providing unit
33: Stress survey unit
310: Image analysis unit 320: Computation unit
330: Stress calculation unit 340: Estimation unit
3100: Stress management server 3200: User terminal
410: Image analysis unit 420: Computation unit
430: Stress calculation unit 440: Estimation unit
450: Care program providing unit 460: Stress survey unit

What is claimed is:

1. A method for measuring and managing stress performed by an apparatus for measuring and managing stress comprising at least one processor, comprising:
outputting survey items on a screen according to an interaction in which a camera recording button is input;
generating a stress and internal vulnerability test result data based on response contents to the survey items;
extracting a blood flow location from a face video captured by a camera, and measuring a first heart rate interval based on first pixel data of extracted location;
analyzing a measurement result of measuring the first heart rate interval and calculating a first heart rate variability;
determining a first stress level based on the first heart rate variability;
extracting a blood flow location from a finger video captured by the camera, and measuring a second heart rate interval based on second pixel data of extracted location;
analyzing a measurement result of measuring the second heart rate interval and calculating a second heart rate variability;
determining a second stress level based on the second heart rate variability;
outputting a stress measurement result, which is a prediction result of resilience and a high-risk group for stress, based on the first heart rate variability, the second heart rate variability, the first stress level, the second stress level, and the stress and internal vulnerability test result data; and
providing a stress management program comprising a plurality of care processes according to the stress measurement result.

2. The method of claim 1, wherein displaying a stress measurement result, which is a prediction result of resilience and a high-risk group for stress, based on the first heart rate variability, the second heart rate variability, the first stress level, the second stress level, and the stress and internal vulnerability test result data comprises:
determining the stress measurement result by analyzing the first heart rate variability, the second heart rate variability, the first stress level, the second stress level, and the stress and internal vulnerability test result data through a machine learning-based model.

3. The method of claim 1, wherein outputting survey items on a screen according to an interaction in which a camera recording button is input comprises:
starting capturing a user using the camera according to the interaction;
outputting the survey items on the screen while the user is being captured; and
terminating capturing of the user when a preset time related to a recording time has elapsed.

4. The method of claim 3, wherein terminating capturing of the user when a preset time related to a recording time has elapsed comprises:
terminating capturing of the user when it is determined that 1 minute has elapsed since the interaction occurred.

5. The method of claim 1, wherein each of the first heart rate variability and the second heart rate variability comprises a time domain index analyzed in a time domain and a frequency domain index analyzed in a frequency domain for heart rate interval data collected for a predetermined time,
the time domain index comprises a SDNN (Standard Deviation of Normal-to-Normal intervals) and a RMSSD (Root Mean Square of Successive Differences between normal heartbeats), and
the frequency domain index represents ratios occupied by VLF (Very low frequency: 0.0033 to 0.04 Hz band), LF (Low frequency: 0.04 to 0.15 Hz band), and HF (High frequency: 0.15 to 0.4 Hz band) in a frequency domain by frequency-converting a heart rate interval data.

6. The method of claim 5, wherein the SDNN is determined based on a mathematical formula
$$SDNN = \sqrt{\frac{1}{N - 1} {{\sum }_{i = 1}^{N} [ Mean ( NN ) - {NN}_{i} ]}^{2}} ,$$
where the i and the N are mathematical formula natural numbers, and the NN is the first heart rate interval or the second heart rate interval, and
the RMSSD is determined based on a mathematical formula
$$RMSSD = \sqrt{\frac{1}{N - 2} {{\sum }_{i = 2}^{N} [ Mean ( NN ) - {NN}_{i - 1} ]}^{2}} .$$

7. The method of claim 1, wherein providing a stress management program comprising a plurality of care processes according to the stress measurement result comprises:
determining a care plan in which the plurality of care processes are provided—the care plan comprises first plan information for determining at least some care processes among the plurality of care processes and second plan information for determining an order in which the at least some care processes are provided—;
providing the at least some care processes according to a result of the care plan;
outputting a sub-stress measurement result according to each of the at least some care processes when each of the at least some care processes is terminated;
calculating a change value between a first sub-stress measurement result measured first and a second sub-stress measurement result measured last when all of the at least some care processes are provided; and
outputting the calculated change value.

8. The method of claim 7, wherein providing the at least some care processes according to a result of the care plan comprises:
starting capturing of a user using the camera in conjunction with an operation of providing the at least some care processes, and
wherein calculating a change value between a first sub-stress measurement result measured first and a second sub-stress measurement result measured last when all of the at least some care processes are provided comprises:
terminating capturing of the user when all of the at least some care processes are provided.

9. The method of claim 7, wherein determining a care plan in which the plurality of care processes are provided comprises:
determining whether a past change value determined within a preset previous time period from a current point in time exists; and
determining the care plan based on the past change value when it is determined that the past change value exists.

10. The method of claim 9, wherein determining the care plan based on the past change value when it is determined that the past change value exists comprises:
determining the care plan based on the past change value when the past change value is equal to or greater than a predetermined threshold; and
determining the care plan without using the past change value when the past change value is less than the predetermined threshold.

11. The method of claim 10, wherein determining the care plan based on the past change value when the past change value is equal to or greater than a predetermined threshold comprises:
determining a past care provision history from which the past change value was calculated; and
determining the care plan based on the past care provision history.

12. An apparatus for measuring and managing stress, comprising:
a stress survey unit configured to display survey items on a screen according to an interaction in which a camera recording button is input, and generate stress and internal vulnerability test result data based on response contents to the survey items;
an image analysis unit configured to extract a blood flow location from a face video captured by a camera, measure a first heart rate interval based on first pixel data of extracted location, extract a blood flow location from a finger video captured by the camera, and measure a second heart rate interval based on second pixel data of extracted location;
a computation unit configured to analyze a measurement result of measuring the first heart rate interval and calculate a first heart rate variability, and analyze a measurement result of measuring the second heart rate interval and calculate a second heart rate variability;
a stress calculation unit configured to determine a first stress level based on the calculated first heart rate variability, and determine a second stress level based on the calculated second heart rate variability;
an estimation unit configured to output a stress measurement result, which is a prediction result of resilience and a high-risk group for stress, based on the first heart rate variability, the second heart rate variability, the first stress level, the second stress level, and the stress and internal vulnerability test result data; and
a care program providing unit configured to provide a stress management program comprising a plurality of care processes according to the stress measurement result.
